(19) United States
(12) Patent Application Publication
(10) Pub. No.: US 2004/0023861 A1
(43) Pub. Date: Feb. 5, 2004

(54) Cysteinyl leukotriene receptor 2 (cysl t2)

(76) Inventors: Diane Joan Cousens, Stevenage (GB); Filippo Volpe, Stevenage (GB); Diane Michele Ignar, Durham, NC (US)

Correspondence Address: DAVID J LEVY, CORPORATE INTELLECTUAL PROPERTY, GLAXOSMITHKLINE, FIVE MOORE DR., PO BOX 13398, RESEARCH TRIANGLE PARK, NC, 27709-3398, US

(21) Appl. No.: 10/182,605

(22) PCT Filed: Feb. 12, 2001
(86) PCT No.: PCT/GB01/00560

(30) Foreign Application Priority Data
Feb. 10, 2000 (GB) 0003079.1

(51) Int. Cl. A61K 38/17; C07H 21/04; C07K 14/705; C12P 21/02; C12N 5/06

(52) U.S. Cl. 514/12; 530/350; 536/23.5; 435/69.1; 435/320.1; 435/325

(57) ABSTRACT

The present invention provides an isolated cysteinyl leukotriene receptor polypeptide comprising

(i) the amino acid sequence of SEQ ID NO: 2 or

(ii) a variant thereof which is capable of binding a leukotriene; or

(iii) a fragment of (i) or (ii) which is capable of binding a leukotriene.

Also provided is a polynucleotide encoding a cysteinyl leukotriene receptor polypeptide, methods for identifying modulators of a cysteinyl leukotriene receptor polypeptide. Such modulators are useful in the treatment of respiratory diseases such as asthma, chronic obstructive pulmonary disease (COPD) and allergic rhinitis and cardiovascular diseases such as cardiac arrhythmia, myocardial ischaemia, atherosclerosis and heart failure.

### FIELD OF THE INVENTION

The present invention relates to cysteinyl leukotriene-receptor polypeptides.

### BACKGROUND OF THE INVENTION

Leukotrienes are a family of eicosinoids which form part of a much larger group of compounds synthesised from arachadonic acid. Phospholipid undergoes metabolic degradation to form arachidonic acid which is further metabolised to produce leukotrienes such as LTB₄, LTD₄, LTE₄, LTC₄ and LTF₄. There are two main classes of leukotriene receptor, the cysteinyl leukotriene receptors and BLT receptors. Two leukotriene receptors have been cloned. CNsLT1 which is activated bv LTD₄ and BLT which is activated by LTB₄.

The BLT receptor responds to LTB₄. LTB₄ is produced mainly by macrophages and neutrophils and stimulates neutrophil chemotaxis, enhances neutrophil-endothelial cell interactions and stimulates neutrophil activation leading to degranulation and the release of mediators, enzymes and superoxides.

The cysteinyl leukotriene receptors respond to LTD₄, LTE₄, LTC₄ and LTF₄, however the occurrence in vivo of LTF₄ is unclear. Cysteinyl leukotrienes contract airway smooth muscle, increase microvascular permability, stimulate mucus secretion, decrease mucociliar, clearance and recruit eosinophils into the airways.

CysLT1 receptor-specific leukotriene receptor antagonists, such as montelukast, zafirlukast and pranlukast are currently used to control bronchoconstriction and inflammation in asthmatic patients. CysLT1 is mainly detected in lung smooth muscle cells, macrophages, spleen and peripheral blood lymphocytes and has not been detected in heart.

### SUMMARY OF THE INVENTION

A novel cysteinyl leukotriene-receptor referred to herein as HIPHUM0000007 is now provided which is a screening target for the identification and development of novel pharmaceutical agents, including modulators of a cysteinyl leukotriene-receptor. The cysteinyl leukotriene-receptor is shown to be primarily expressed in heart spleen, adrenal, placenta, in peripheral blood mononuclear cells including monocytes, neutrophilsand eosinophils. Further the gene encoding the novel cysteinyl leukotriene-receptor has been mapped to a chromosomal region linked to asthma. Novel pharmaceutical agents identified using HIPHUM0000007 may be used in the therapeutic treatment and/or prophylaxis of disorders such as cardiovascular diseases such as cardiac arrhythmia, myocardial ischaemia, atherosclerosis and heart failure, lung diseases such as asthma, chronic obstructive pulmonary disease (COPD) and allergic rhinitis. Additionally, these agents may also be used in the therapeutic treatment and/or propylaxis of immune deficiency disorder, AIDS, rheumatoid arthritis, multiple sclerosis leukaemia, myasthenia gravis, graves disease, systemic lupus ernthematosus, inflammatonr bowel disease, encephalomyelitis psoriasis, atopic dermatitis, septic shock, stroke and ischaemia reperfusion injury.

Accordingly, the present invention provides an isolated cysteinyl leukotriene-receptor polypeptide comprising

(i) the amino acid sequence of SEQ ID NO: 2, or

(ii) a variant thereof which is capable of binding a leukotriene;

(iii) a fragment of (i) or (ii) which is capable of binding a leukotriene.

According to another aspect of the invention there is provided a polynucleotide encoding a polypeptide of the invention which polynucleotide includes a sequence comprising:

(a) the nucleic acid sequence of SEQ ID NO: 1 and/or a sequence complementary thereto:

(b) a sequence which hybridises under stringent conditions to a sequence as defined in (a);

(c) a sequence that is degenerate as a result of the genetic code to a sequence as defined in (a) or (b); or

(d) a sequence having at least 60% identity to a sequence as defined in (a), (b) or (c).

The invention also provides:

an expression vector which comprises a polynucleotide of the invention and which is capable of expressing a polypeptide of the invention:

a host cell comprising an expression vector of the invention:

a method of producing a polypeptide of the invention which method comprises maintaining a cell line of the invention under conditions suitable for obtaining expression of the polypeptide and isolating the said polypeptide:

an antibody specific for a polypeptide of the invention:

a method for identification of a substance that modulates cysteinyl leukotriene-receptor activity, which method comprises contacting a polypeptide of the invention with a test substance and monitoring for cysteinyl leukotriene-receptor activity;

a substance which stimulates or modulates HIPHUM0000007 receptor activity and which is identifiable by the method referred to above;

a method of treating a subject having a disorder that is responsive to HIPHUM0000007 receptor stimulation or modulation, which method comprises administering to said patient an effective amount of a substance of the invention; and

use of a substance that stimulates or modulates cysteinyl leukotriene-receptor activity in the manufacture of a medicament for the treatment or prophylaxis of a disorder that is responsive to stimulation or modulation of HIPHUM0000007 receptor activity.

Preferably the disorder is selected from asthma, chronic obstructive pulmonary disease (COPD), allergic rhinitis,cardiac arrhythmia, myocardial ischaemia, atherosclerosis and heart failure.

### BRIEF DESCRIPTION OF THE FIGURES

FIG. 1 shows the relative mRNA expression levels of HIPHUM0000007 in normal human tissues.

FIG. 2 shows the relative mRNA expression levels of HIPHUM0000007 in immune cells.

FIG. 3 shows the relative mRNA expression levels of HIPHIM0000007 in normal and diseased human lung tissues.

FIG. 4A shows the results of a NFAT luciferase reporter gene assay to determine the responsiveness of HIPHUTM0000007 stably expressed in chinese hamster ovary cells containing the NFAT reporter gene (CHONFAT cells) to the leukotrienes, LTC₄, LTD₄ and LTE₄. FIG. 4B shows the results of a luciferase assay to determine the responsiveness of CysLT1 stably expressed in CHONFAT cells to LTC₄, LTD₄ and LTE₄.

FIG. 5 shows the results of a fluoresence Imaging Plate Leader (FLIPR) assay to determine the responsiveness of HIPHUM 0000007 (FIG. 5A) or CysLT1 (FIG. 5B) stably expressed in CHONFAT cells to the leukotrienes. LTC₄, LTD₄ and LTE₄.

FIG. 6 shows the concentration dependent antagonistic activity of BayU9773 on CysLT1 (LT1) and HIPHUM 0000007 (LT2) activity in response to 20 nM LTD₄ as measured by a NFAT luciferase reporter assay (FIG. 6A) and a FLIPR assay (FIG. 6B).

FIG. 7 shows the concentration dependent antagonist activity of the CysLT1 antagonists. GW483100X (Montelukast), GR34820X, AH23134X and GR138714X (Zafirlukast) on HIPHUM 0000007 (FIG. 7A) and CysLT1 (FIG. 7B) activity in response to 20 nM LTD₄ as determined using a NFAT luciferase reporter gene assay.

FIG. 8 shows the concentration dependent antagonist activity of the CysLT1 antagonists. GW483100X (Montelukast), GR34820X, AH23134X and GR138714X (Zafirlukast), on HIPHUM 0000007 (FIG. 8A) and CysLT1 (FIG. 8B) activity in response to 20 nM LTD₄ as determined using a FLIPR assay.

FIG. 9 shows the effect of intravenous injection of various concentrations of LTD₄ on the mean blood pressure of anesthetized rats over time.

FIG. 10 shows the effect of oral administration of 3 μmol/kg of various CysLT1 antagonists on the change in blood pressure resulting from intravenous administration of 0.1 μmol/kg LTD₄ to anaesthetized rats. Each antagonist was administered 10 minutes before LTD₄ injection.

FIG. 11 shows the effect of intravenous administration of 0.3 μmol/kg LTD₄-induced change in blood pressure is anaesthetized rats. Each antagonist was administered 5 minutes prior to the LTD₄ intravenous injection.

FIG. 12 shows the marked rhvthm disturbances resulting from repeated administration of LTD₄ to anaesthetised rats. These arrhythmias were observed repeatedly at various LTD₄ doses (in the range of 40 to 60 nmol/kg). FIGS. 12A and B shows venticular ectopic bits and salvos. FIGS. 12C and 12D show atrio-ventricular blocks.

### BRIEF DESCRIPTION OF THE SEQUENCES

SEQ ID NO: 1 is the DNA and amino acid sequence of human protein HIPHUM0000007 and its encoding DNA.

SEQ ID NO: 2 is the amino acid sequence alone of HIPHUM0000007.

### DETAILED DESCRIPTION OF THE INVENTION

Throughout the present specification and the accompanying claims the words “comprise” and “include” and variations such as “comprises”, “comprising”, “includes” and “including” are to be interpreted inclusively. That is, these words are intended to convey the possible inclusion of other elements or integers not specifically recited, where the context allows.

The present invention relates to a human cysteinyl leukotriene-like receptor, referred to herein as HIPHUM0000007, and variants thereof. Sequence information for HIPHUM0000007 is provided in SEQ ID NO: 1 (nucleotide and amino acid) and in SEQ ID NO: 2. The polypeptides of the invention consist essentially of the amino acid sequence of SEQ ID NO: 2 or of a variant of that sequence.

Polypeptides of the invention may be in a substantially isolated form. The term “isolated” is intended to convey that the polypeptide is not in its native state, insofar as it has been purified at least to some extent or has been synthetically produced, for example by recombinant methods. It will be understood that the polypeptide may be mixed with carriers or diluents which will not interfere with the intended purpose of the polypeptide and still be regarded as substantially isolated. The term “isolated” therefore includes the possibility of the polypeptide being in combination with other biological or non-biolozical material, such as cells, suspensions of cells or cell fragments, proteins peptides, expression vectors, organic or inorganic solvents, or other materials where appropriate but excludes the situation where the polypeptide is in a state as found in nature.

A polypeptide of the invention may also be in a substantially purified form, in which case it will generally comprise the polypeptide in a preparation in which more than 50%, e.g. more than 80%, 90%, 95% or 99%, by weight of the polypeptide in the preparation is a polypeptide of the invention. Routine methods, can be employed to purify and/or synthesise the proteins according to the invention. Such methods are well understood by persons skilled in the art, and include techniques such as those disclosed in Sambrook el al, Molecular Cloning: a Laboratory Manual. 2^{nd} Edition, CSH Laboratory Press (1989), the disclosure of which is included herein in its entirety by way of reference.

The term “variant” refers to a polypeptide which has the same essential character or basic biological functionality as HIPHUM0000007. The essential character of HIPHUM0000007 can be defined as follows: HIPHUM0000007 is a cysteinyl leukotriene-receptor. Preferably a variant polypeptide is one which binds to the same ligand as HIPHUM0000007. Preferably the polypeptide has leukotriene binding activity. Examples of such leukotrienes include LTB₄, LTC₄,LTD₄, LTE₄, and LTF₄. More preferably the polypeptide binds LTC₄,LTD₄ and LTE₄. Most preferably the polypeptide binds LTC₄ with ahigher affinity than LTD₄ than LTE₄. A polypeptide having the same essential character as HIPHUM0000007 may be identified by monitoring for binding of a leukotriene. A full length protein is preferably one which includes a seven transmembrane region. Preferably, the full-length receptor may couple to G-protein to mediate intracellular responses. Preferably the receptor is activatived by LTC₄, LTD₄ and LTE₄. More preferably, LTC₄ exhibits a higher efficacy than LTD₄ which in turn is more efficacious than LTE₄.

In another aspect of the invention, a variant is one which does not show the same function as HIPHUM0000007 but is one which inhibits the basic function of HIPHUM0000007. For example, a variant polypeptide is one which inhibits leukotriene-mediated activity of HIPHUM0000007, for example by binding to a ligand of HIPHUM0000007 such as LTC₄ to prevent the ligand binding to HIPHUM0000007.

Typically, polypeptides with more than about 65% identity preferably at least 80% or at least 90% and particularly preferably at least 95% at least 97% or at least 99% identity with the amino acid sequences of SEQ ID NO: 2 are considered as variants of the proteins. Such variants may include allelic variants and the deletion modification or addition of single amino acids or groups of amino acids within the protein sequence, as long as the peptide maintains the basic biological functionality of the HIPHUJM0000007 receptor.

Amino acid substitutions may be made for example from 1, 2 or 3 to 10, 20 or 30 substitutions. The modified polypeptide generally retains activity as a HIPHUM0000007 receptor. Conservative substitutions may be made, for example according to the following Table. Amino acids in the same block in the second column and preferably in the same line in the third column may be substituted for each other.

| | | |
|---|---|---|
| ALIPHATIC | Non-polar | GAP |
| | | ILV |
| | Polar-uncharged | CSTM |
| | | NQ |
| | Polar-charged | DE |
| | | KR |
| AROMATIC | | HFWY |

Shorter polypeptide sequences are within the scope of the invention. For example, a peptide of at least 20 amino acids or up to 50, 60, 70, 80, 100, 150 or 200 amino acids in length is considered to fall within the scope of the invention as long as it demonstrates the basic biological functionality of HIPHUM0000007. In particular. but not exclusively, this aspect of the invention encompasses the situation when the protein is a fragment of the complete protein sequence and may represent a ligand-binding region (N-terminal extracellular domain) or an effector binding region (C-terminal intracellular domain). Such fragments can be used to construct chimeric receptors preferable with another 7-transmembrane receptor, more preferably with another member of the family of cysteinyl leukotriene-receptors. Such fragments of HIPHUM0000007 or a variant thereof can also be used to raise anti-HIPHUM0000007 antibodies. In this embodiment the fragment may comprise an epitope of the HIPHUM0000007 polypeptide and may otherwise not demonstrate the ligand binding or other properties of HIPHUM0000007.

Polypeptides of the invention may be chemically modified, e.g. post-translationally modified. For example, they may be glycosylated or comprise modified amino acid residues. They may also be modified by the addition of histidine residues to assist their purification or by the addition of a signal sequence to promote insertion into the cell membrane. Such modified polypeptides fall within the scope of the term “polypeptide” of the invention.

The invention also includes nucleotide sequences that encode for HIPHUM0000007 or a variant thereof as well as nucleotide sequences which are complementary thereto. The nucleotide sequence may be RNA or DNA including genomic DNA, synthetic DNA or cDNA. Preferably the nucleotide sequence is a DNA sequence and most preferably, a cDNA sequence. Nucleotide sequence information is provided in SEQ ID NO: 1. Such nucleotides can be isolated from human cells or synthesised according to methods well known in the art, as described by way of example in Sambrook et al.

Typically a polynucleotide of the invention comprises a contiguous sequence of nucleotides which is capable of hybridizing under selective conditions to the coding sequence or the complement of the coding sequence of SEQ ID NO: 1.

A polynucleotide of the invention can hydridize to the coding sequence or the complement of the coding sequence of SEQ ID NO: 1 at a level significantly above background. Background hybridization may occur, for example, because of other cDNAs present in a cDNA library. The signal level generated by the interaction between a polynucleotide of the invention and the coding sequence or complement of the coding sequence of SEQ ID NO: 1 is tropically at least 10 fold, preferably at least 100 fold, as intense as interactions between other polynucleotides and the coding sequence of SEQ ID NO: 1. The intensity of interaction may be measured, for example, by radiolabelling the probe, e.g. with ³²P. Selective hybridisation may typically be achieved using conditions of medium to high stringency (for example, 2×SSC 0.15M sodium chloride and 0.015M sodium citrate at about 50° C. to about 60° C.). However, such hybridisation may be carried out under any suitable conditions known in the art (see Sambrook et al (1989) Molecular Cloning: A Labaratory Manual). For example, if high stringency is required suitable conditions include from 0.1 to 0.2×SSC at 60° C. up to 65° C. If lower stringency is required suitable conditions include 2×SSC at 60° C.

The coding sequence of SEQ ID NO: 1 may be modified by nucleotide substitutions, for example from 1, 2 or 3 to 10, 25, 50 or 100 substitutions. The polynucleotide of SEQ ID NO: 1 may alternatively or additionally be modified by one or more insertions and/or deletions and/or by an extension at either or both ends. A polynucleotide may include one or more introns, for example may comprise genomic DNA. Additional sequences such as signal sequences which may assist in insertion of the polypeptide in a cell membrane may also be included. The modified polynucleotide generally encodes a polypeptide which has HIPHUM0000007 receptor activity. Alternatively, a polynucleotide encodes a ligand-binding portion of a polypeptide or a polypeptide which inhibits HIPHUM0000007 activity. Degenerate substitutions may be made and/or substitutions may be made which would result in a conservative amino acid substitution when the modified sequence is translated, for example as shown in the Table above.

A nucleotide sequence which is capable of selectively hybridizing to the complement of the DNA coding sequence of SEQ ID NO: 1 will generally have at least 60%, at least 70%, at least 80%, at least 90%, at least 95%, at least 98% or at least 99% sequence identity to the coding sequence of SEQ ID NO: 1 over a region of at least 20, preferably at least 30, for instance at least 40, at least 60, more preferably at least 100 contiguous nucleotides or most preferably over the full length of SEQ ID NO: 1.

For example the UWGCG Package provides the BESTFIT program which can be used to calculate homology (for example used on its default settings) (Devereux et al (1984) Nucleic Acids Research 12. p387-395). The PILEUP and BLAST alaorithms can be used to calculate homology or line up sequences (typically on their default settings), for example as described in Altschul S. F. (1993) J Mol Evol 36:290-300; Altschul. S. F et al (1990) J Mol Biol 21 5:403-10.

Software for performing BLAST analyses is publicly available through the National Centre for Biotechnology Information (http://ww.ncbi.nlm.nih.gov/). This algorithm involves first identifying high scoring sequence pair (HSPs) by identifying short words of length W in the query sequence that either match or satisfy some positive-valued threshold score T when aligned with a word of the same length in a database sequence. T is referred to as the neighbourhood word score threshold (Altschul et al, supra). These initial neighbourhood word hits act as seeds for initiating searches to find HSPs containing them. The word hits are extended in both directions along each sequence for as far as the cumulative alignment score can be increased. Extensions for the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment. The BLAST program uses as defaults a word length (W) of 11. the BLOSUM62 scoring matrix (see Henikoff and Henikoff (1992) Proc. Natl. Acad. Sci. USA 89: 10915-10919) alignments (B) of 50, expectation (E) of 10, M=5, N=4, and a comparison of both strands.

The BLAST algorithm performs a statistical analysis of the similarity between two sequences see e.g., Karlin and Altschul (1993) Proc. Natl. Acad. Sci. USA 90: 5873-5787. One measure of similarity provided by the BLAST algorithm is the smallest sum probability (P(N)), which provides an indication of the probability by which a match between two nucleotide or amino acid sequences would occur by chance. For example, a sequence is considered similar to another sequence if the smallest sum probability in comparison of the first sequence to the second sequence is less than about 1 preferably less than about 0.1, more preferably less than about 0.01, and most preferably less than about 0.001.

Any combination of the above mentioned degrees of sequence identity and minimum sizes may be used to define polynucleotides of the invention, with the more stringent combinations (i.e. higher sequence identity over longer lengths) being preferred. Thus, for example a polynucleotide which has at least 90% sequence identity over 25, preferably over 30 nucleotides forms one aspect of the invention, as does a polynucleotide which has at least 95% sequence identity over 40 nucleotides.

The nucleotides according to the invention have utility in production of the proteins according to the invention, which may take place in vitro, in vivo or ex vivo. The nucleotides may be involved in recombinant protein synthesis or indeed as therapeutic agents in their own right, utilised in gene therapy techniques. Nucleotides complementary to those encoding HIPHUM0000007, or antisense sequences, may also be used in gene therapy.

Polynucleotides of the invention may be used as a primer, e.g. a PCR primer, a primer for an alternative amplification reaction, a probe e.g. labelled with a revealing label by conventional means using radioactive or non-radioactive labels, or the polynucleotides may be cloned into vectors.

Such primers, probes and other fragments will preferably be at least 10, preferable at least 15 or at least 20, for example at least 25, at least 30 or at least 40 nucleotides in length. They will typically be up to 40, 50, 60, 70, 100 or 150 nucleotides in length. Probes and fragments can be longer than 150 nucleotides in length, for example up to 200, 300, 400, 500, 600, 700 nucleotides in length, or even up to a few nucleotides, such as five or ten nucleotides, short of the coding sequence of SEQ ID NO: 1.

The present invention also includes expression vectors that comprise nucleotide sequences encoding the proteins or variants thereof of the invention. Such expression vectors are routinely constructed in the art of molecular biology and may for example involve the use of plasmid DNA and appropriate initiators, promoters, enhancers and other elements, such as for example polyadenylation signals which may be necessary, and which are positioned in the correct orientation, in order to allow for protein expression. Other suitable vectors would be apparent to persons skilled in the art. By way of further example in this regard we refer to Sambrook et al, 1989.

Poly nucleotides according to the invention may also be inserted into the vectors described above in an antisense orientation in order to provide for the production of anti sense RNA. Anti sense RNA or other antisense polynucleotides may also be produced by synthetic means. Such antisense polynucleotides may be used as test compounds in the assays of the invention or may be useful in a method of treatment of the human or animal body by therapy.

Preferably, a polynucleotide of the invention or for use in the invention in a vector is operably linked to a control sequence which is capable of providing for the expression of the coding sequence by the host cell, i.e. the vector is an expression vector. The term “operably linked” refers to a juxtaposition wherein the components described are in a relationship permitting them to function in their intended manner. A regulatory sequence, such as a promoter, “operably linked” to a coding sequence is positioned in such a way that expression of the coding sequence is achieved under conditions compatible with the regulatory sequence.

The vectors may be for example, plasmid, virus or phage vectors provided with a origin of replication, optionally a promoter for the expression of the said polynucleotide and optionally a regulator of the promoter. The vectors may contain one or more selectable marker genes, for example an ampicillin resistence gene in the case of a bacterial plasmid or a resistance gene for a fungal vector. Vectors may be used in vitro, for example for the production of DNA or RNA or used to transfect or transform a host cell, for example, a mammalian host cell. The vectors may also be adapted to be used in vivo, for-example in a method of gene therapy.

Promoters and other expression regulation signals may be selected to be compatible with the host cell for which expression is designed. For example, yeast promoters include S. cerevisiae GAL4 and ADH promoters, S. pombe nmt1 and adh promoter. Mammalian promoters include the metallothionein promoter which can be induced in response to heavy metals such as cadmium. Viral promoters such as the SV40 large T antigen promoter or adenovirus promoters mayt also be used. All these promoters are readily available in the art.

Mammalian promoters, such as β-actin promoters, may be used. Tissue-specific promoters are especially preferred. Viral promoters may also be used, for example the Moloney murine leukaemia virus long terminal repeat (MMLV LTR), the rous sarcoma virus (RSV) LTR promoter, the SV40 promoter, the human cytomegalovirus (CMV) IE promoter, adenovirus, HSV promoters (such as the HSV IE promoters), or HPV promoters, particularly the HPV upstream regulatory region (URR). Viral promoters are readily available in the art.

The vector may further include sequences flanking the polynucleotide giving rise to polynucleotides which comprise sequences homologous to eukaryotic genomic sequences preferably mammalian genomic sequences, or viral genomic sequences. This will allow the introduction of the polynucleotides of the invention into the genome of eukaryotic cells or viruses by homologous recombination. In particular, a plasmid vector comprising the expression cassette flanked by viral sequences can be used to prepare a viral vector suitable for delivering the polynucleotides of the invention to a mammalian cell. Other examples of suitable viral vectors include herpes simplex viral vectors and retroviruses, including lentiviruses, adenoviruses, adeno-associated viruses and HPV viruses. Gene transfer techniques using these viruses are known to those skilled in the art. Retrovirus vectors for example may be used to stably integrate the polynucleotide giving rise to the polynucleotide into the host genome. Replication-defective adenovirus vectors by contrast remain episomal and therefore allow transient expression.

The invention also includes cells that have been modified to express the HIPHUM0000007 polypeptide or a variant thereof. Such cells include transient, or preferably stable higher eukaryotic cell lines, such as mammalian cells or insect cells, using for example a baculovirus expression system, lower eukaryotic cells, such as yeast or prokaryotic cells such as bacterial cells. Particular examples of cells which may be modified by insertion of vectors encoding for a polypeptide according to the invention include mammalian HEK293T, CHO, HeLa and COS cells. Preferably the cell line selected will be one which is not only stable, but also allows for mature glycosylation and cell surface expression of a polypeptide. Expression may be achieved in injected oocytes. A polypeptide of the invention may be expressed in cells of a transgenic non-human animal, preferably a mouse. A transgenic non-human animal expressing a polypeptide of the invention is included within the scope of the invention. A polypeptide of the invention may also be expressed in Xenopus laevis oocytes or melanophores, in particular for use in an assay of the invention.

According to another aspecl the present invention also relates to antibodies (either polyclonal or preferably monoclonal antibodies, chimeric, single chain and Fab fragments) which have been raised by standard techniques and are specific for a polypeptide of the invention. Such antibodies are, for example, useful in purification, isolation or screening methods involving immunoprecipitation techniques, as tools to further elucidate the function of HIPHUM0000007 or a variant thereof, or indeed as therapeutic agents in their own right.

Antibodies may also be raised against specific epitopes of the proteins according to the invention. Such antibodies may be used to block ligand binding to the receptor. An antibody, or other compounds, “specifically binds” to a protein when it binds with preferential or high affinity to the protein for which it is specific but does not substantially bind, not bind or binds with only low affinity to other proteins. A variety of protocols for competitive binding or immunoradiometric assays to determine the specific binding capability of an antibody are well known in the art (see for example Maddox et al. J. Exp. Med. 158. 1211-1226, 1993). Such immunoassays typically involve the formation of complexes between the specific protein and its antibody and the measurement of complex formation.

Antibodies of the invention may be antibodies to human polypeptides or fragments thereof. For the purposes of this invention, the term “antibody”, unless specified to the contrary, includes fragments which bind a polypeptide of the invention. Such fragments include Fv, F(ab′) and F(ab′)₂ fragments, as well as single chain antibodies. Furthermore, the antibodies and fragment thereof may be chimeric antibodies, CDR-grafted antibodies or humanised antibodies.

Antibodies may be used in a method for detecting polypeptides of the invention in a biological sample, which method comprises:

I providing an antibody of the invention:

II incubating a biological sample with said antibody under conditions which allow for the formation of an antibody-antigen complex; and

III determining whether antibody-antigen complex comprising said antibody is formed.

A sample may be for example a tissue extract, blood, serum and saliva. Antibodies of the invention may be bound to a solid support and/or packaged into kits in a suitable container along with suitable reagents, controls, instructions, etc. Antibodies may be linked to a revealing label and thus may be suitable for use in methods of in vivo HIPHUM0000007 imaging.

Antibodies of the invention can be produced by any suitable method. Means for preparing and characterising antibodies are well known in the art, see for example Harlow and Lane (1988) “Antibodies: A Laboratory Manual”, Cold Spring Harbor Laboratory Press, Cold Spring Harbor, N.Y. For example, an antibody may be produced by raising antibody in a host animal against the whole polypeptide or a fragment thereof, for example an antigenic epitope thereof, herein after the “immunogen”.

A method for producing a polyclonal antibody comprises immunising a suitable host animal, for example an experimental animal, with the immunogen and isolating immunoglobulins from the animal's serum. The animal may therefore be inoculated with the immunogen, blood subsequently removed from the animal and the IgG fraction purified.

A method for producing a monoclonal antibody comprises immortalising cells which produce the desired antibody. Hybridoma cells may be produced by fusing spleen cells from an inoculated experimental animal with tumour cells (Kohler and Milstein (1975) Nature 256, 495-497).

An immortalized cell producing the desired antibody may be selected by a conventional procedure. The hybridomas may be grown in culture or injected intraperitoneally for formation of ascites fluid or into the blood stream of an allogenic host or immunocompromised host. Human antibody may be prepared by in vitro immunisation of human lymphocytes, followed by transformation of the lymphocytes with Epstein-Barr virus.

For the production of both monoclonal and polyclonal antibodies, the experimental animal is suitably a goat, rabbit, rat or mouse. If desired, the immunogen may be administered as a conjugate in which the immunogen is coupled, for example via a side chain of one of the amino acid residues, to a suitable carrier. The carrier molecule is typically a physiologically acceptable carrier. The antibody obtained may be isolated and, if desired, purified.

An important aspect of the present invention is the use of poly peptides according to the invention in screening methods. The screening methods may be used to identify substances that bind to cysteinyl leukotriene-receptors and in particular which bind to HIPHUM0000007 such as a ligand for the receptor. Screening methods may also be used to identify agonists or antagonists which may modulate cysteinyl leukotriene-receptor activity, inhibitors or activators of HIPHUM0000007 activitv and/or agents which up-regulate or down-regulate HIPHUM0000007 expression. Preferably such screening rncthods may be used to identify modulators of LTC₄-mediated HIPHUM000007 activity. For example. a screening method may be used to identify a substance which mimics, inhibits or enhances LTC₄-mediatcd activity.

In a preferred embodiment, the invention provides a scrcening method for identifying a substnce which acts as an antagonist of both HIPHUM0000007 activity and CysLT1 activity. A modulator of HIPHUM0000007 activity identiied by a method described herein may be tested for modulator activity at the CysLT1 receptor. Alternatively, a candidate modulator may first be screened for CysLT1 modulator activity and then tested for HIPHUM0000007 activity.

Any suitable format may be used for the assay. In general terms such screening methods may involve contacting a polypeptide of the invention uith a test substance and monitoring for binding of the test substance or measuring receptor activity or may involve incubating a polypeptide of the invention with a test substance and then detecting modulation of leukotriene-activity, preferably LTC₄ activity, at the receptor. In a preferred aspect, the assay is a cell-based assay. Preferably the assay may be carried out in a single well of micotitre plate. Assay formats whicb allow high throughput screening are preferred.

Modulator activity can be determined by contacting cells expressing a polypeptide of the invention with a substance under investigation and by monitoring an effect mediated by the polypeptides. The cells expressing the polypeptide may be in vitro or in vivo. The polypeptide of the invention may be naturally or recombinantly expressed. Preferably, the assay is carried out in vitro using cells expressing recombinant polypeptide. Preferably, control experiments ae carried out on cells which do not express the polypeptide of the invention to establish whether the observed responses are the result of activation of the polypeptide.

The binding of a test substance to a polypeptide of the invention can be determined directly. For example, a radiolabelled test substance can be incubated with the polypeptide of the invention and binding of the test substance to the polypeptide can be monitored. Typically, the radiolabelled test substance can be incubated with cell membranes containiine the polypeptide until equilibrium is reached. The membranes can then be separated from a non-bound test substance and dissolved in scintillation fluid to allow the radioactive content to be determined by scintillation counting. Non-specific binding of the test substance may also be determined by repeating the experiment in the presence of a saturating concentration of a non-radioactive ligand.

Assays may be carried out be incubating a cell expressing a receptor in accordance with the invention with a test substance and monitoring chemotaxis of the cells in response to a ligand, such as LTC₄, LTD₄ or LTE₄.

Assays may be carried out using cells expressing HIPHUM0000007, and incubating such cells with the test substance optionally in the presence of HIPHUM0000007 ligand. Altenatively an antibody may be used to complex HIPHUM0000007 and thus mediate HIPHUM0000007-activity. Test substances may then be added to assess the effect on such activity. Cells expressing HIPHUM0000007 constitutively may be provided for use in assays for HIPHHUM0000007 function. Such constitutively expressed HTPHUM0000007 may demonstrate HIPHUM0000007 activity in the absence of ligand binding. Additional test substances may be introduced in any assay to look for inhibitors of ligand binding or inhibitors of HIPHUM0000007-mediated activity.

In preferred aspects, a host cell is provided expressing the receptor polypeptide and containing a G-protein coupled pathway responsive reporter construct. The host cell is treated with a substance under test for a defined time. The expression of the reporter gene, such as SP alkaline phosphatase or luciferase is assayed. The assay enables determination of whether the compound modulates the induction of the G-protein coupled pathway by the cysteinyl leukotriene-receptor in target cells.

Assays may also be carried out to identify modulators of receptor shedding. A polypeptide of the invention can be cleaved from the cell surface. Shedding the receptor would act to down regulate receptor signalling. Thus, cell based assays may be used to screen for compounds which promote or inhibit receptor-shedding.

Assays may also be carried out to identify substances which modify HIPHUM0000007 receptor expression for example substances which up or down regulate expression. Such asseys may be carried out for example by using antibodies for HIPHUM0000007 to monitor levels of HIPHUM0000007 expression. Other assays which can be used to monitor the effect of a test substance on HIPHUM0000007 expression include using a reporter gene construct driven by the HIPHUM0000007 regulatory sequences as the promoter sequence and monitoring for expression of the reporter polypeptide. Further possible assays could utilise membrane fractions from overexpression of HIPHUM0000007 receptor either in X. laevis oocytes or cell lines such as HEK293, CHO, COS7 and HeLa cells and assessmnent of displacement of a radiolabelled cysteinyl leukotriene-ligand.

Additional control experiments may be carried out. Assays may also be carried out using known ligands of other cysteinyl leukotriene-receptors such as CysLT1 to identify ligands which are specific for polypeptides of the invention. Preferably, the assays of the invention are carried out under conditions which would result in G-protein coupled pathway mediated activity in the absence of the test substance, to identify inhibitors or activators of cysteinyl leukotriene-like receptor mediated activity, or agents which inhibit ligand-induced cysteinyl leukotriene-like receptor activity. An assay of the invention may be carried out using a known cysteinyl leukotriene-agonist or cysteinyl leukotriene-antagonist to provide a comparison with a compound under test.

Typically, receptor activity can be monitored indirectly for example by measuring a G_{q}-coupled readout. G_{q} coupled readout can typically be monitored using an electrophysiological method to determine the activity of G-protein regulated Ca²⁺ or K⁻ channels or by using a fluorescent dye to measure changes in intracellular Ca²⁺ levels. Other methods that can typically be used to monitor receptor activity involved measuring levels of or activity of GTPγS or cAMP.

Xenopus dermal melanophores aggregate or disperse pigment in response to the activation or inhibition of G protein coupled receptors. This feature can be exploited as an assay for receptor activation or inhibition if a specific G protein coupled receptor is exogenously expressed.

HIPHUM0000007 receptor is likely to couple to G protein wiih consequent hydrolysis of GTP. Accumulation of a labelled GTP stable analogue can be measured utilising membrane fractions from overexpression of HIPHUM0000007 receptor eiher in X. laevis oocytes or cell lines such as HEK293, CHO, COS7 or HeLa cells on cxposure to agonist ligand.

G protein coupled receptors have been shown to activate MAPK signalling pathways. Cell lines overexpressing the cysteinyl leukotriene-like receptor with MAPK reporter ganes may be utilised as assays for receptor activation or inhibition. The cysteinyl leukotriene-receptor of the invention may be heterologously expressed in modified yeast strains containing multiple reporter genes, such as FUS1-HIS3 and FUS1-lacZ, each linked to an endogenous MAPK cascade-based signal transduction pathway. This pathway is normally linked to pheromone receptors, but can be coupled to foreign receptors by replacement of the yeast G protein with yeast/mammalian G protein chimeras. Strains may also contain two further gene deletions, of SST2 and FAR1, to potentiate the assay. Ligand activation of the heterologous receptor can be monitored using the reporter genes, for example either as cell growth in the absence of bistidine or with a substrate of beta-galactosidase (lacZ).

Further possible assays could utilise membrane fraction from overexpression of HIPHUM0000007 rceptor either in X. laevis oocytes or cell lines such as HEK293, CHO, COS7, HeLa and displacement of radiolabelled leukotriene ligand, i.e. [³H]LTD₄ or other leukotriene, can be readily assessed (Yokomizo T. et al, 1997, Nature, 387, 620-624).

Suitable test substances which can be tested in the above assays include combinatorial libraries, defined chemical entities, peptide and peptide mimetics, oligonucleotides and natural product libraries, such as display (e.g. phage display libraries) and antibody products.

Typically, organic molecules will be screened, preferably small organic molecules which bave a molecular weight of from 50 to 2500 daltons. Candidate products can be biomolecules including, saccharides, fatty acids, steroids, purines, pyrimidines. derivatives, structural analogs or combinations thereof. Candidate agents are obtained from a wide variety of sources including libraries of synthetic or natural compounds. Knowvn pbarmacological agents may be subjected to directed or random chemical modifications, such as acylation, alklyation, esterification, amidification, etc. to produce structural analogs.

Test substances may be used in an initial screen of, for example, 10 substances per rcaction, and the substances of these batches whicb show inhibition or activation tested individually. Test substances may be used at a concentration of from 1 nM. to 1000 μM, preferably from 1 μM to 100 μM, more preferably from 1 μM to 10 μM. Preferably, the activity of a test substance is compared to the activity shown by a known activator or inhibitor. A test substance which acts as an inhibitor may produce a 50% inhibition of activity of the receptor. Alternatively a test substance which acts as an activator may produce 50% of the maximal activity produced using a known activator.

Another aspect of tbe present iuvention is the use of polynucleotides encoding the HIPHUM0000007 polypeptides of the invention to identify mutations in HIPHUM0000007 genes which may be implicated in human disorders. Identification of such mutations may be used to assist in diagnosis or susceptibility to such disorders and in assessing the physiology of such disorders. Polynucleotides may also be used in hybridisation studies to monitor for up or down regulation of HIPHUM0000007 expression. Polynucleotides such as SEQ ID NO: 1 or fragments thereof may be used to identify allelic variants, genomic DNA and species variants.

The present invention provides a method for detecting variation in the expressed products encoded by HIPHUM0000007 genes. This may comprise determining the level of HIPHUM0000007 expressed in cells or determining specific alterations in the expressed product. Sequences of interest for diagnostic purposes include, but are not limited to, the conserved portions as identified by sequence similarity and conservation of intron/exon structure. The diagnosis may be performed in conjunction wth kindred studies to determine whether a mutation of interest co-segregates with disease phenotype in a family.

Diagnostic procedures may be performed on polynucleotides isolated from an individual or alternatively, may be performed in situ directly upon tissue sections (fixed and/or frozen) of patient tissue obtained from biopsies or resections, such that no nucleic acid purification is necessary. Appropriate procedures are described in, for example, Nuovo. G. J., 1992. “PCR In Situ Hybridization: Protocols And Applications”, Raven Press. NY). Such analysis techniques include, DNA or RNA blotting analyses, single stranded conformational polymorphism analyses, in situ hbridization assays, and polymerase chain reaction analyses. Such analyses may reveal both quantitative aspects of the expression pattern of a HIPHUM0000007, and qualitative aspects of HIPHUM0000007 expression and/or composition.

Alternative diagnostic methods for the detection of HIPHUM0000007 nucleic acid molecules may involve their amplification e.g. by PCR (the experimental embodiment set forth in U.S. Pat. No. 4,683,202), ligase chain reaction (Barany, 1991 Proc Natl. Acad. Sci. U.S.A 88:189-193), self sustained sequence replication (Guatelli et al. 1990. Proc. Natl. Acad. Sci. U.S.A 87:1874-1878), transcriptional amplification system (Kwoh et al, 1989, Proc. Natl. Acad. Sci. 15 U.S.A 86:1173-1177), Q-Beta Replicase (Lizardi et al., 1988. Bio/Technology 6:1197) or any other nucleic acid amplificaiion method, followed by the detection of the amplified molecules using techniques well known to those of skill in the art. These detection schemes are especially useful for the detection of nucleic acid molecules if such molecules are present in very low numbers.

Particularly suitable diagnostic methods are chip-based DNA technologies such as those described by Hacia et al., 1996, Nature Genetics 14:441-447 and Shoemaker et al., 1996, Nature Genetics 14:450-456. Briefly, these techniques involve quantitative methods for analyzing large nunbers of nucleic acid sequence targets rapidly and accurately. By tagging with oligonucleotides or using fixed probe arrays, one can employ chip technology to segregate target molecules as high density arrarys and screen these molecules on the basis of hybridization.

Following detection, the results seen in a given patient may be compared with a statistically significant reference group of normal patients and patients that have HIPHUM0000007 related pathologies. In this way, it is possible to correlate the amount or kind of HIPHUM0000007 encoded product detected with various clinical states or predisposition to clincal states.

Another aspect of the present invention is the use of the substances that have been identified by screening techniques referred to above in the treatment or prophylaxis of disorders which are responsive to regulation of cysteinyl leukotriene-receptor activity. The treatment may be therapeutic or prophylactic. The condition of a patient suffering from such a disorder can thus be improved.

In particular, such substances may be used in the treatment of respiratory diseases such as asthma, chronic obstructive pulmonary disease (COPD) and allergic rhinitis and cardiovascular diseases such as cardiac arrhythmia, myocardial ischaemia, atherosclerosis and heart failure. Additional disorders that may be treated by such substances include immune deficiency disorder, AIDS, rheumatoid arthritis, multiple sclerosis, leukaemia myasthenia gravis, graves diseases, systemic lupus erythematosus, inflammatory bowel disease, encephalomyelitis, psoriasis. atopic dermatitis, septic shock, stroke and ischaemia reperfusion injury. It is to be understood that mention of these specific disorders is by way of example only and is not intended to be limiting on the scope of the invention as described. In particular, modulators of HIPHUM0000007 function may be administered to treat the conditions mentioned above.

Inhibitors of HIPHUM0000007 activity may be particularly useful in the treatment of respiratory disease and cardiovascular disease. For example, dual antagonists whiclh inhibit both HIPHUM0000007 activity and CysLT1 activity may be useful in treating such diseases. Accordingly; the present invention provides a method of treating a disorder that is responsive to cysteinyl leukotriene receptor modulation, which method comprises administering to said subject a therapeutically effective amount of a substance which inhibits HIPHUM0000007 acivity or HIPHUM0000007 and CysLT1 activity.

Substances identified according to the screening methods outlined above may be formulated with standard pharmaceutically acceptable carriers and/or excipients as is routine in the pharmaceutical art. For example, a suitable substance may be dissolved in physiological saline or water for injections. The exact nature of a formulation upon several factors including the particular substance to be administered and the desired route of administration. Suitable types of formulation are fully described in Remmington's Pharmaceutical Sciences, Mack Publishing Company, Eastern Pennsylvania 17^{th} Ed. 1985, the disclosure of which is included herein of its entirety by way of reference.

The substances may be administered by enteral or parenteral routes such as via oral, buccal, anal, pulmonary, intravenous, intra-arterial, intramusculer, intraperitoneal, topical or other appropriate administraton routes.

A therapeutically effctive amount of a modulator is administered to a patient. The dose of a modulator may be determined according to various parameters, especially according to the substance used; the age, weight and condition of the patient to be treated; the route of administration; and the required regimen. A physician will be able to determine the required route of administration and dosage for any particular patient. A typical daily dose is from about 0.1 to 50 mg per kg of body weight, according to the activity of the specific modulator, the age, weight and conditions of the subject to be treated, the type and severity of the degeneration and the frequency and route of administration. Preferably, daily dosage levels are from 5 mg to 2 g.

Nucleic acid encoding HIPHUM0000007 or variant thereof which inhibits binding of a leukotriene may be administered to the mammal. Nucleic acid, such as RNA or DNA, and preferably, DNA, is provided in the form of a vector, such as the polynucleotides described above, whice may be expressed in the cells of the mammal.

Nucleic acid encoding the polypeptide may be administered to the animal by any available technique. For example, the nucleic acid may be introduced by injection, preferably intradermally, subcutaneously or intramuscularly. Alternatively, the nucleic acid may be delivered directly across the skin using a nucleic acid delivery device such as particle-mediated gene delivery. The nucleic acid may be administered topically to the skin, or to mucosal surfaces for example by intranasal, oral, intravaginal or intrarectal administration.

Uptake of nucleic acid constructs may be enhanced by several known transfection techniques, for example those including the use of transfection agents. Examples of these agents includes cationic agents, for example, calcium phosphate and DEAE-Dextran and lipofectants, for example, lipofectam and transfectam. The dosage of the nucleic acid to be administered can be altered. Typically the nucleic acid is administered in the range of 1 pg to 1 mg, preferably to 3 pg to 10 μg nucleic acid for particle mediated gene delivery and 10 μg to 1 mg for other routes.

The following Examples illustrate the invention.

### EXAMPLE 1

### Characterisation of HIPHUM0000007 Sequence

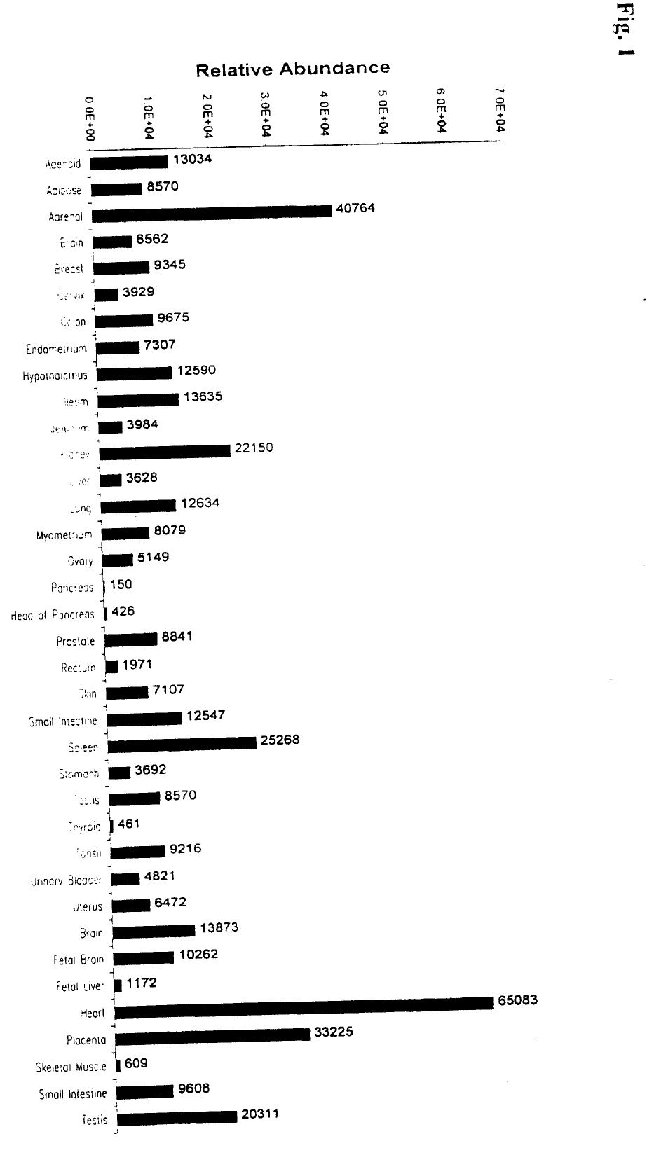

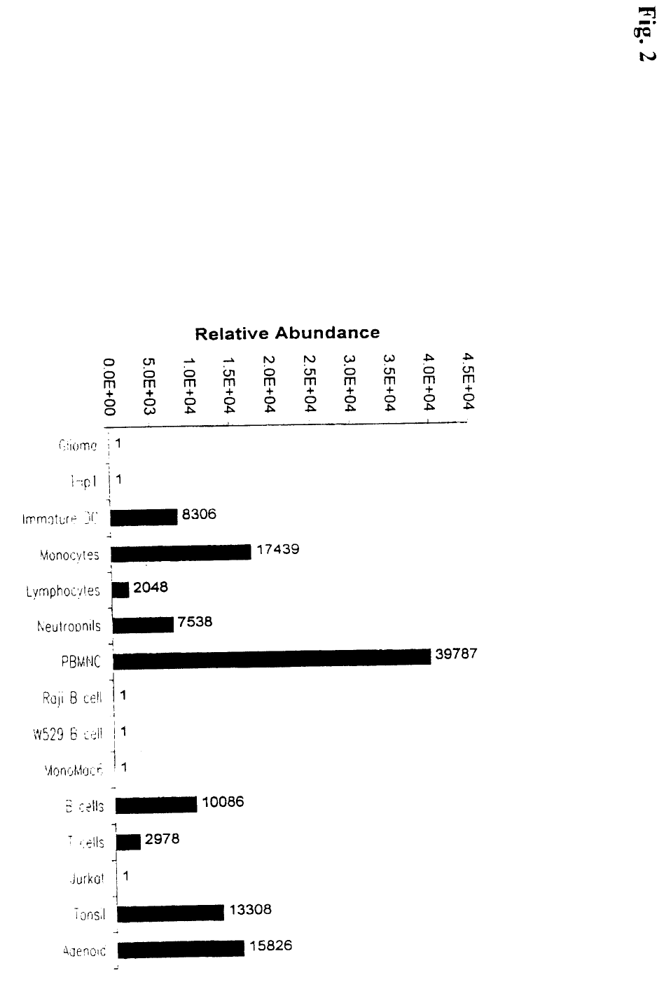

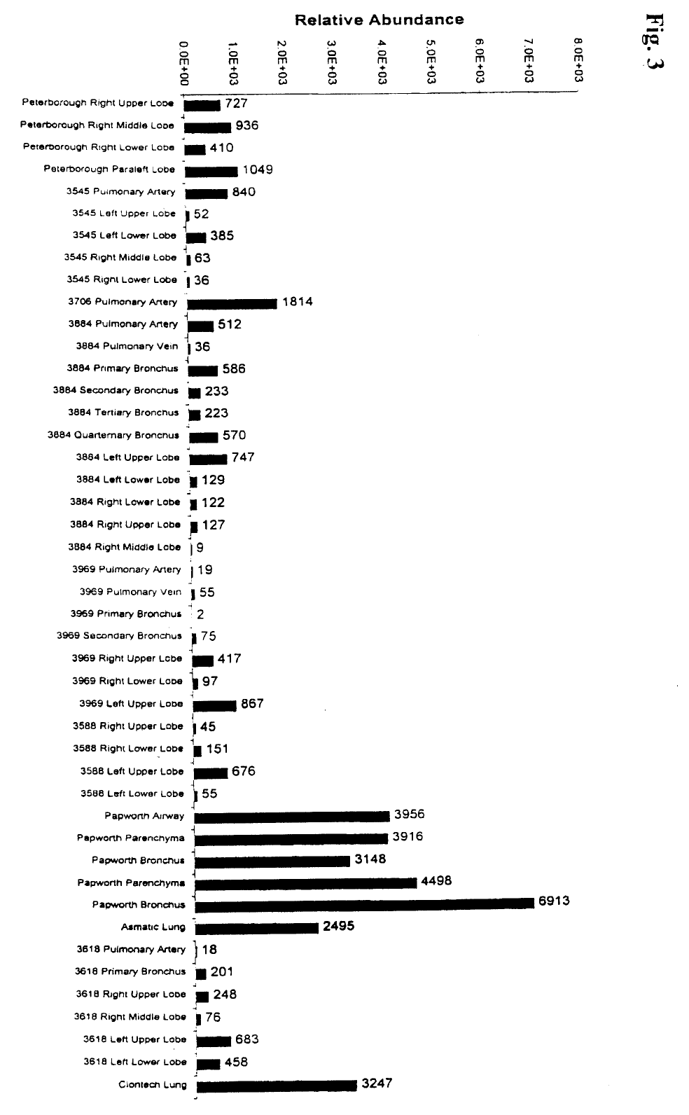

A cysteinyl leukotriene-receptor polypeptide, designated HIPHUM0000007 has been identified. The nucleotide and amino acid sequence of the receptor have been determined. These are set out below in SEQ ID NOS. 1 and 2. Suitable primers and probes were designed and used to analyse tissue by Taqman™ analysis. The results by Taqman™ analysis expression in nonnal human tissues, in immune cells and in normal and diseased lung tissues are shown in FIGS. 1, 2 and 3 respectively. The cysteinyl leukotriene-receptor is shown to be primarily expressed in heart, spleen, adrenal gland, placenta, in peripheral blood mononuclear cells including monocytes and eosinophils. The novel cysteinyl leukotriene-receptor may be up-regulated or down-regulated in diseased lung.

The chromosomnal localisation was also mapped. Human HIPHUM0000007 has been mapped to chromosome 13q14. Chromosome 13q14 has been linked to asthma.

### EXAMPLE 2

### Expression of HIPHUM0000007 in Xenopus Oocytes

Adult female Xenopus laevis (Blades Biologicals) were anaesthetised using 0.2% tricaine (3-aminobenzoic acid ethyl ester), killed and the ovaries rapidly removed. Oocytes were then de-folliculated by collagenase digestion (Sigma type I, 1.5 mg ml⁻¹) in divalent cation-free OR2 solution (82.5 mM NaCl, 2.5 mM KCl, 1.2 mM NaH₂PO₄, 5 mM HEPES; pH 7.5 at 25° C.). Single stage V and VI oocytes were transfee,d to ND96 solution (96 mM NaCl, 2 mM KCl, 1 mM MgCl₂, 5 mM HEPES, 2.5 mM sodium pyruvate; pH 7.5 at 25° C.) which contained 50 μg ml⁻¹ gentamycin and stored at 18° C.

The cysteinyl leukotriene-like rceptor (in pcDNA₃, Invitrogen) was linearised and transcribed to RNA using T7 (Promega Wizard kit). m′G(5′)pp(5′)GTP capped cRNA was injected into oocytes (20-50 ng per oocyte) and whole-cell currents in response to LTD₄ and LTC₄ were recorded using two-microelectrode voltage-clamp (Geneclamp amplifier, Axon instruments Inc.) 3 to 7 days post-RNA injection. Microelectrodes had a resistance of 0.5 to 2MΩ when filled with 3 M KCl.

### EXAMPLE 3

### Mammalian Cell Line Construction

IRES expression vectors containing the CMV promoter were used to stably transfect CysLT1 and HIPHUM0000007 in HEK293 and CHO lines. The same constructs were then used to generate a stable CHO cell line which had an NFAT-luciferase gene stably integrated.

### EXAMPLE 4

### Reporter Gene Experiments to Determine Agonist Activity

Agonist activity was measured in Chinese hamster ovary (CHO) cells containinig the NFAT reporter gene which, upon stimulation, produced luciferase. A cell line stably expressing the human CysLT1 receptor and a ceil line stably expresing HIPHUM0000007 were tested.

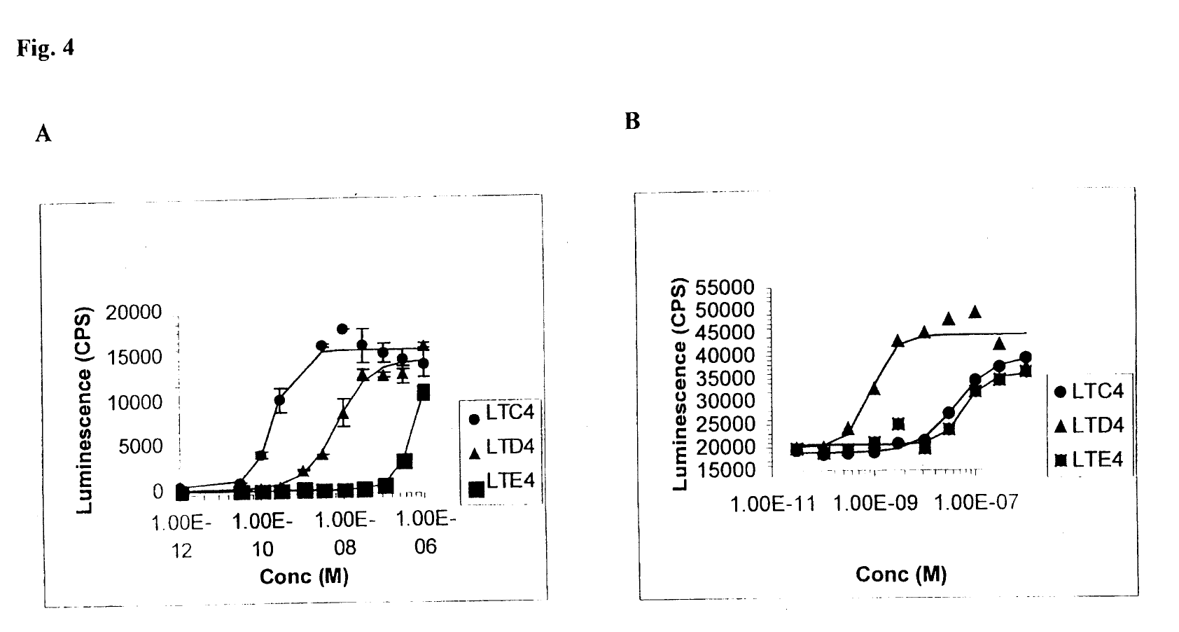

Celis were plated out in 96 well plates in culture medium and grown in an incubator at 37° C. for 48 hours. When confluent, culture medium was removed and the cells were quiesced by adding culture mediun without any growth factors. After 24 hours quiescence, agonists were added to the cells at a concentration range of approximately 10⁻¹² to 10⁻⁶ M. The cells were then incubated with the agonist for 5 hours at 37° C. Luciferase reporter gene assay kit (Packard) was used to measure luciferase production. The fluorescence of luciferase was measured using a plate reader. The results of experiments to measure the activity of LTC₄, LTD₄ and LTE₄ at HIPHUM0000007 and at the CysLT1 receptor are shown in FIG. 4. The rank order of agonists at CysLT1 was LTD₄>LTE₄=LTC₄ whilst at HIPHUM0000007 the agonist rate order was LTC₄>LTD₄>LTE₄.

### EXAMPLE 5

### Fluorescence Imaging Plate Reader (FLIPR) Experiments to Determine Agonist Activity

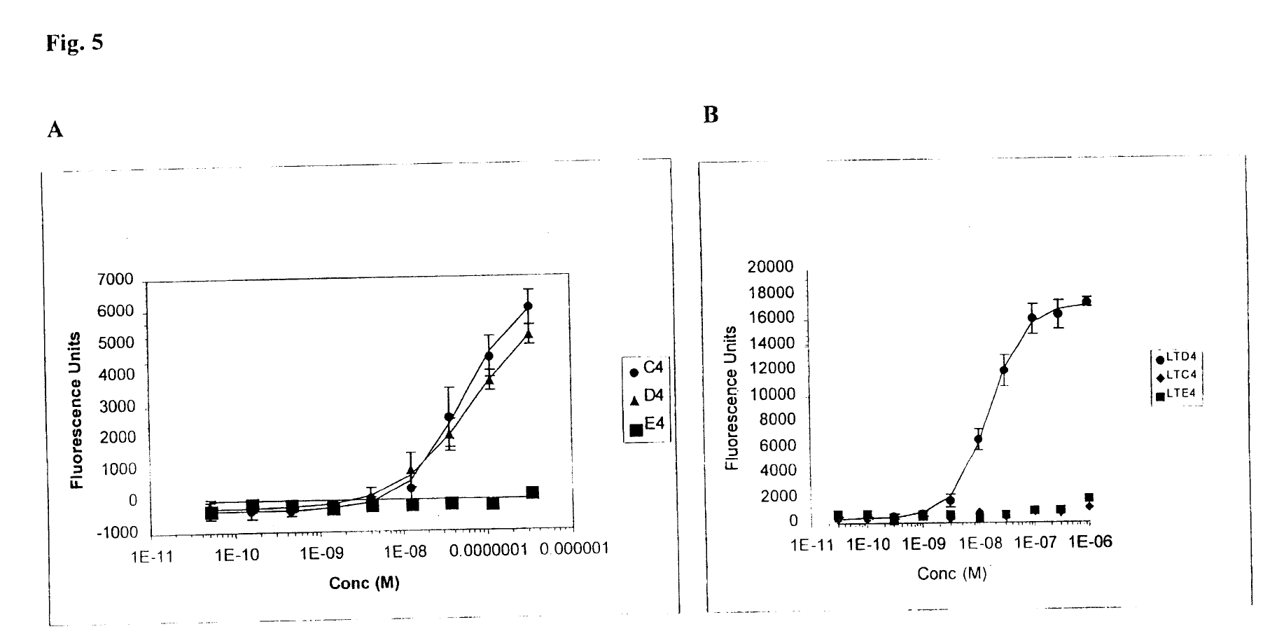

Agonist activity was measured in the cell lines described in Example 4. Cells were grown to confluence in 96 well black plates and loaded with Tyrodes buffer (145 mM NaCl, 10 mM glucose, 2.5 mM KCl, 1 mM MgCl₂, 1.5 mM CaCl₂, 10 mM HEPES, pH 7.4) containing 2 μm Fluo-4 AM, a calcium-selective, fluorescent indicator and 2.5 mM probenacid (a transport inhibitor). The cells were incubated with this loading dye for 75 minutes after which time excess Fluo-4 was washed from the cells and replaced with Tyrodes buffer. Agonist was added to the cells at a concentration range of approximately 10⁻¹¹ to 10⁻⁶ M. Calcium mobilisation in response to HIPHUM0000007 or CysLT1 receptor activity was measured using FLIPR detection of calcium bound to Fluo-4. Readings were taken at 2 minute intervals. The concentration dependent calcium mobilisation in response to activation of HIPHUM0000007 and CysLT1 by LTC₄, LTD₄ and LTE₄ is shown in FIG. 5.

### EXAMPLE 6

### Effect of CysLT1 Antagonist, BayU9773

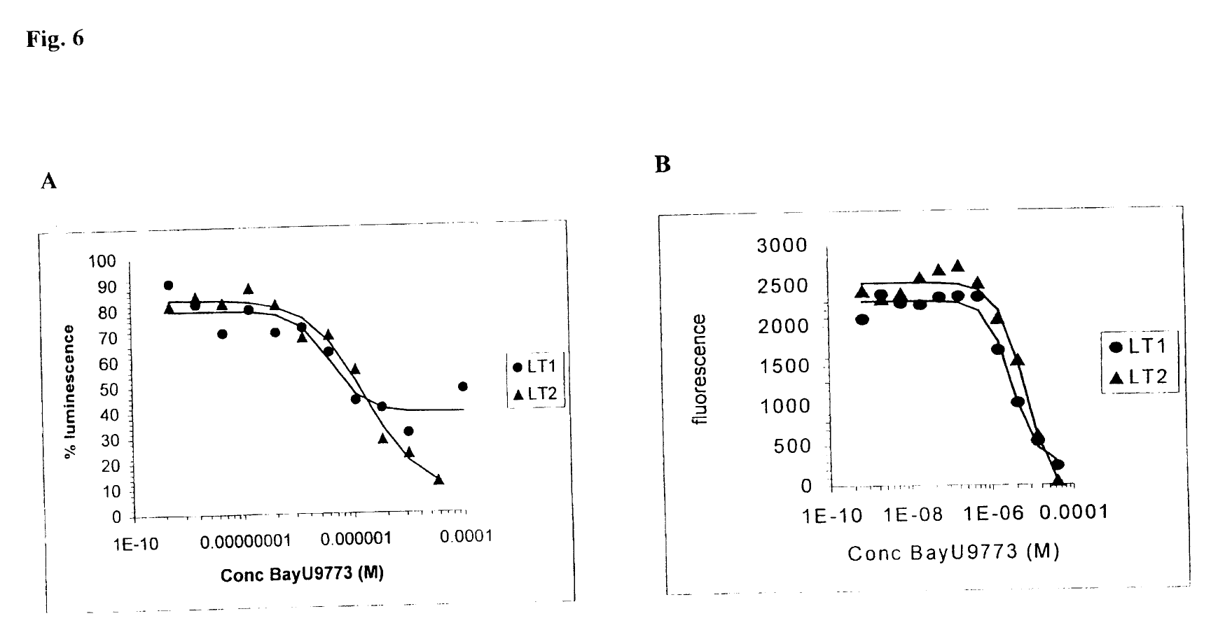

The antagonist activity of BayU9773 at HIPHUM0000007 and CysLT1 receptors was determined by measuring the concentration dependent inhibition of 20 nM LTD₄. Bay U9773 was added 5 minutes prior to LTD₄ in reporter gene experiments and 7 minutes prior tc LTD₄ in FLIPR experiments. FIG. 6A shows the results of the reporter gene assay and FIG. 6B shows the results of the FLIPR assay BayU9773 has a low potency at both CysLT1 and at HIPHUM0000007

### EXAMPLE 7

### Effect of CysLT1 Antagonists

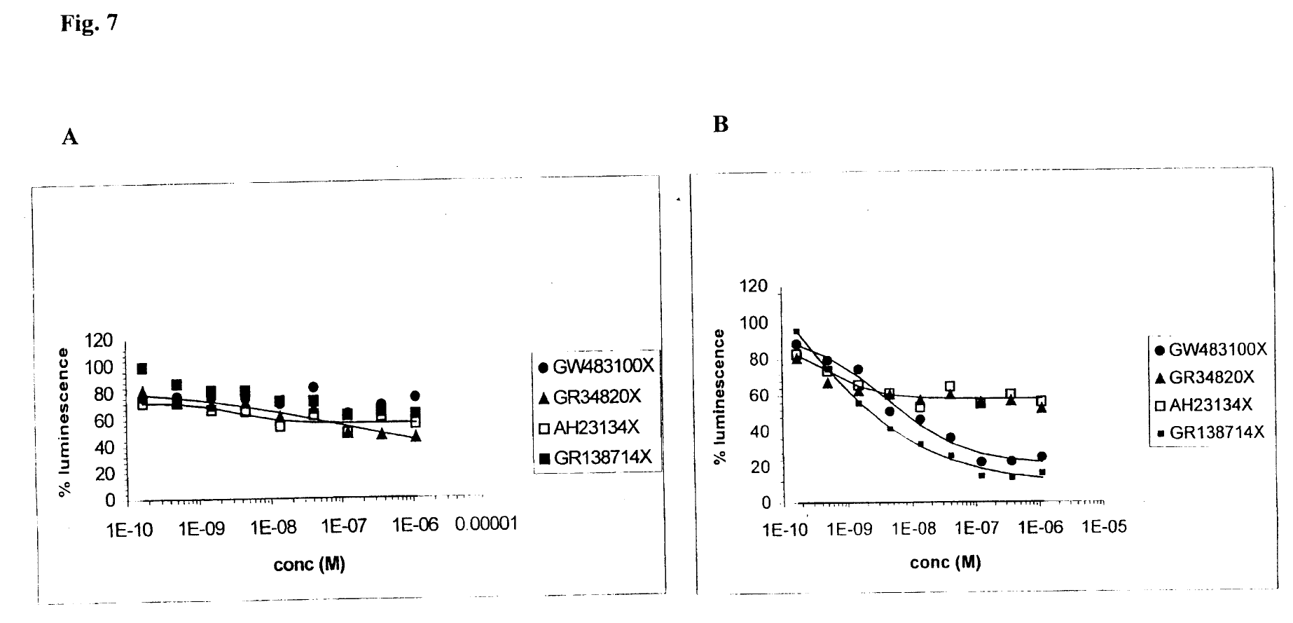

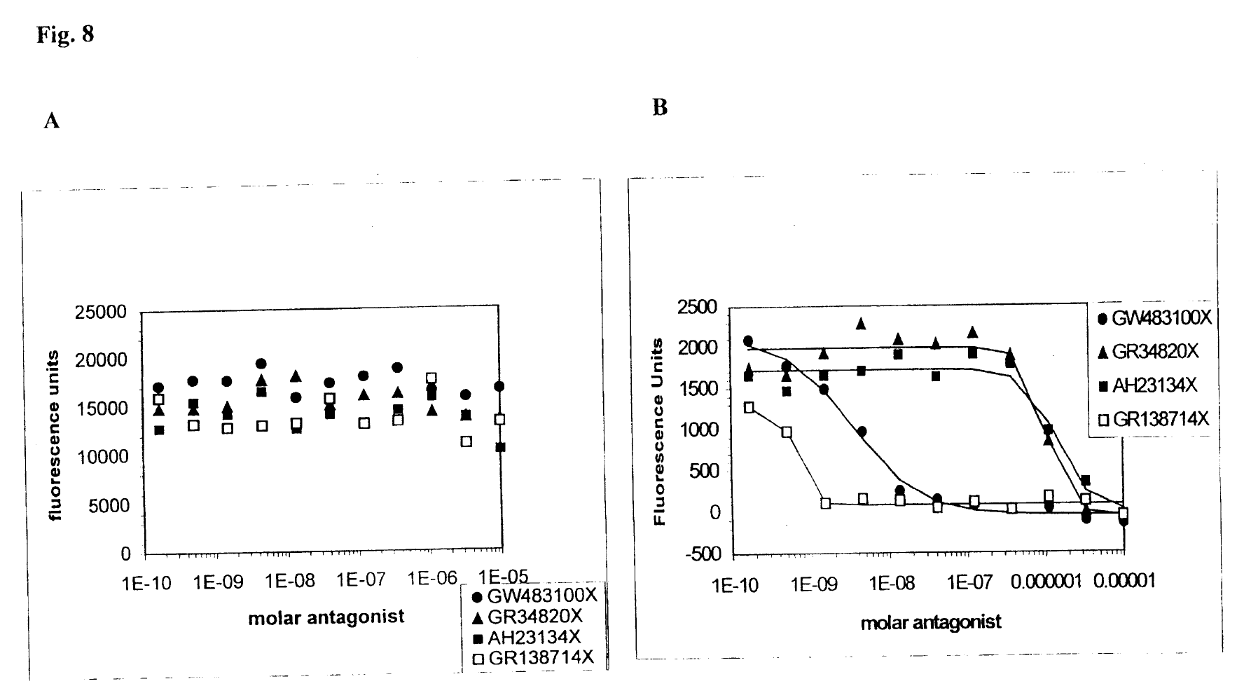

The antagonist activity of known CysLT1 antagonists, Montelukast (GW483100X), Zafirlukast (GR138714X), GP34820X and AH23134, at HIPHUM0000007 and CysLT1 receptors was determined by measuring the concentration dependent inhibition of the receptors in response to stimulation with 20 nM LTD₄. Each antagonist was added 5 minutes prior to LTD₄ addition in reporter gene experiments and 7 minutes prior to LTD₄ addition in FLIPR experiments. FIG. 7 shows the results of the reporter gene assay and FIG. 8 shows the results of the FLlPR assay. All FOUR antagonists were inactive at HIPHUM0000007 but acted as antagonists at CysLT1.

### EXAMPLE 8

### Screening for Compounds Which Exhibit Protein Modulating Activity

Mammalian cells, such as HEK293, CHO and COS7 cells over-expressing the protein of choice are generated for use in the assay. 96 and 384 well plate, high throughput screens (HTS) are employed using fluorescence based calcium indicator molecules, including but not limited to dyes such as Fura-2. Fura-Red, Fluo 3 and Fluo 4 (Molecular Probes). Secondary screening involves the same technology. Tertiary screens involve the study of modulators in rat, mouse and guinea-pig models of disease relevant to the target.

A brief screening assay protocol is as follows:

Mammalian cells stably over-expressing the protein are cultured in black wall, clear bottom, tissue culture coated 96 or 384 well plates with a volume of 100 μl cell culture medium in each well 1 to 3 days before use in a FLIPR (Fluorscence Imaging Plate Reader—Molecular Devices). Cells are incubated with 2 μM FLUO-4AM at 30° C. in 5% CO₂ for 75 mins and then washed once in Tyrodes buffer (145 mM NaCl, 10 mM glucose, 2.5 mM KCl, 1 mM MgCl₂, 1.5 mM CaCl₂, 10 mM HEPES pH 7.4) containing 3 mM probenacid.

Basal fluorescence (11,000-15,000 FIU) is determined prior to substance additions. The protein is activated upon the addition of a known agonist. Activation results in an increase in intracellular calcium which can be measured directly in the FLIPR. For antagonist studies, substances are preincubated with the cells for 7 minutes following dye loading and washing and fuorescence is measured for 4 minutes. Agonists are then added and cell fluorescence monitored for a further 1 minute.

### EXAMPLE 9

### Effect of LTD₄ on Hemodynamics and Electrocardigram (ECG0 Parameters

Adult male wistar rats (270-300 g) were used. On arrival in the animal care unit, rats were housed with free access to food and water. Room temperature (19-20° C.), relative humidity (55-60%) and light (switched on 7:00am-7:00pm) were maintained constant. An adaptation period of at least 6 days was respected before experimentation.

Aninals (n=3-4) were anesthetized by administration of pentobarbital (50/60 mg/kg, i.v.). Then, animals were placed on a heatcd (36° C.) operating table and ventilated with room air (Harvard respirator, 50-52 str/min, 7 ml/kg tidal volume). Systemic arterial blood pressure was monitored routinely from the left carotid artery (P23XL, Gould). Body temperature was controlled with a thermistor probe (Digi-sense, Cole-Parmer) and maintained within physiological range throughout the experiment by heating when necessary. A Lead 11 ECG was recorded via needle electrodes to evaluate ECG intervals and heart rate and a corrected QT interval by linear regression (QTL) was calculated according to Todt(13). All parameters were monitored on a Gould thermal array polyrecorder.

LTD₄ was purchased from BioMol (Catalog TEBU N° LD-004) and stored at −80° C. until use. After a 10-min stabilization period, a LTD₄ (dissolved in MeOH/NH4OAc 70/30, pH 5.6) soluition was administered within the penis vein as a bolus at the single dose of 20, 40 and 100 nmol/kg (10, 20 and 50 μg/kg, respectively) using a stock solution at 0.1 mM. The volume of injection was normalized to 1 ml/kg. In rats receiving CysLT1 antagonists as pretreatment, the compounds were previously dissolved in ethanol (2% final concentration) or distilled water and mixed in encapsin 6% for intragastric afministration (single dose of 3 μmol/kg-10 ml/kg total volume). Pretreatment with CysLT1 antagonists was given 10 min before LTD₄ i.v. injection (100 nmol/kg. In a second set of experiments, CysLT1 antagonists were intravenously administrated (300 nmoles/kg) 5 minutes prior LTD₄ i.v. injection. Maximal changes in hemodynamic and ECG parameters were continuously monitored uith IOX and ECG software (EMKA, France).

Results were expressed as mean±SEM Hemodynamic and electrocardiographic variables were compared using Anova followed by multiple comparisons tests (Newmam Keuls or Tukey's test as appropriate). Values of p <0.05 were considered significant.

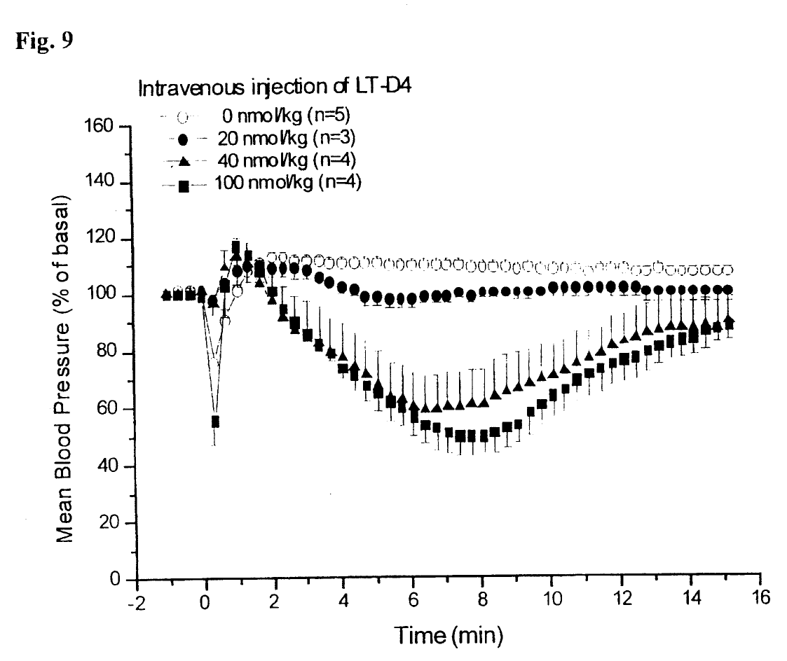

Bolus intravenous admimistration of LTD₄ resulted in a 20-30% reduction in heart rate. This effect was not significantly altered by pretreatment with MK571 (a CysLT1 antagonist) also known as L-660,711. The rsults of LTD₄ injection on blood pressure are shown in FIG. 9.

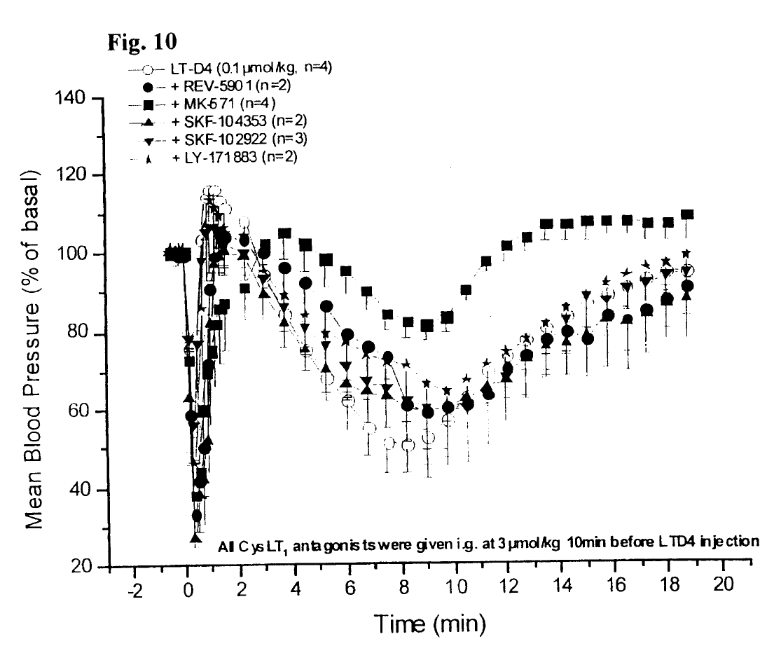

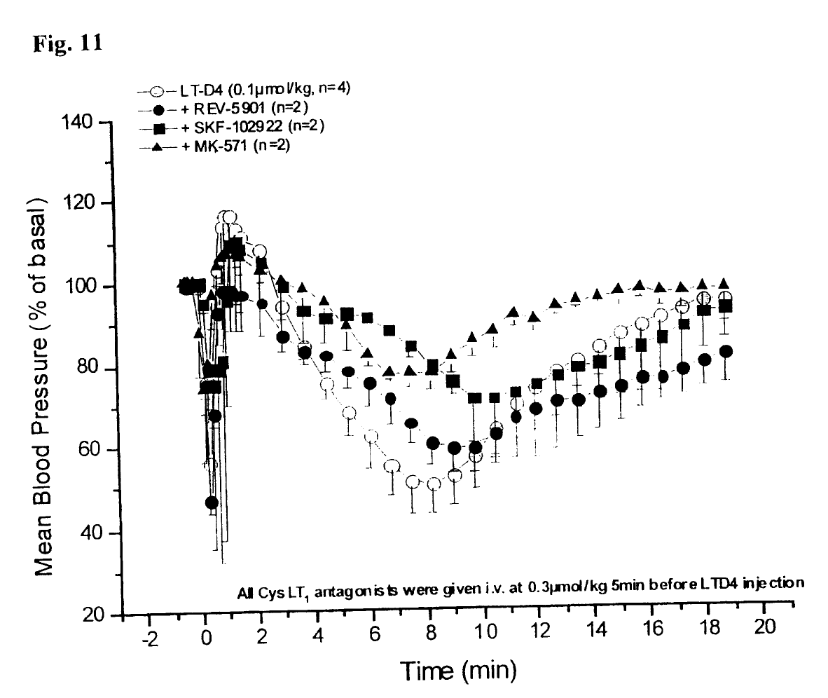

lnjection of LTD₄ also affected blood pressure in a dose-dependent manner. The effect of LTD₄ on blood pressure was also largely unaffected by a panel of CysLT1 antagonsts although there was a modest partial inhibition of LTD₄ response by MK571 on blood pressure. FIG. 10 shows the effect of oral admimistration of 3 μmol/kg of Rev-5901, MK-571, SKF-104353 (pobilukast), SKF-102922 and LY-171883 on LTD₄ mediated effects on blood pressure. FIG. 11 shows the effect of intravenous administration of 0.3 μmol/kg REV-5901, SKF-102922 and MK-571 on the LTD₄ mediated effects on blood pressure.

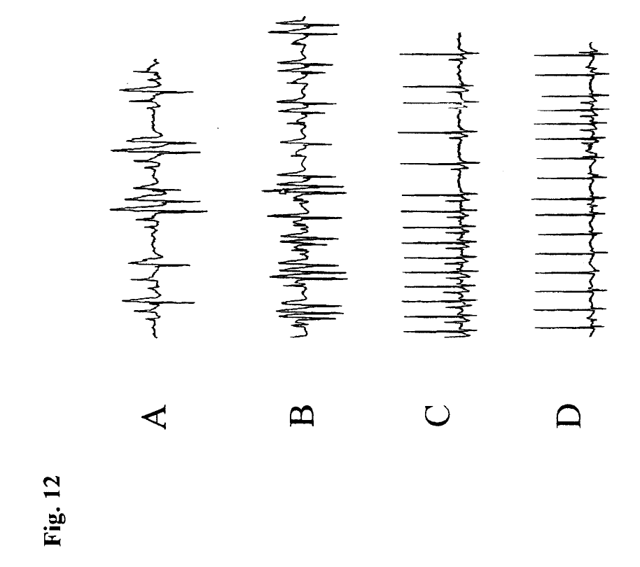

As shown in FIG. 12 repeated administration of LTD₄ induced marked rhythm disturbances. These arrhythmias were observed repeatedly at various LTD₄ doses (in the range of 40 to 60 nmol/kg). They were defined either as ventricular ectopic bits and salvos (panel A and B), or atrio-ventricular blocks (panel C and D).

1. An isolated cysteinyl leukotriene receptor polypeptide comprising
(i) the amino acid sequence of SEQ ID NO: 2 or
(ii) a variant thereof which is capable of binding a leukotriene; or
(iii) a fragment of (i) or (ii) which is capable of binding a leukotriene.

2. A polypeptide according to claim 1 wherein said vector or fragment has a higher affinity for LTC₄ than for LTD₄ than for LTE₄.

3. A polypeptide according to claim 1 or 2 wherein the variant (ii) has at least 80% identity to the amino acid sequence of SEQ ID NO: 2.

4. A polynucleotide encoding a polypeptide according to any one of claims 1 to 3.

5. A polynucleotide according to claim 4 which is a cDNA sequence.

6. A polynucleotide encoding a cysteinyl leukotriene receptor polypeptide which is capable of binding a leukotriene which polynucleotide comprises:
(a) the nucleic acid sequence of SEQ ID NO: 1 and/or a sequence complementary thereto;
(b) a sequence which hybridises under stringent conditions to a sequence as defined in (a);
(c) a sequence that is degenerate as a result of the genetic code to a sequence as defined in (a) or (b); or
(d) a sequence having at least 60% identity to a sequence as defined in (a), (b) or (c).

7. An expression vector comprising a polynucleotide sequence according to any one of claims 4 to 6, which is capable of expressing a polypeptide according to any one of claims 1 to 3.

8. A host cell comprising an expression vector according to claim 7.

9. An antibody specific for a polypeptide according to any one of claims 1 to 3.

10. A method for identification of a substance that modulates cysteinyl leukotriene receptor activity, which method comprises contacting a polypeptide according to any one of claims 1 to 3 with a test substance and monitoring for cysteinyl leukotriene-receptor activity.

11. A method according to claim 10 wherein the polypeptide is expressed in a cell.

12. A substance which modulates cysteinyl leukotriene-receptor activity and which is identifiable by a method according to claim 10 or 11.

13. A substance according to claim 12 which is an inhibitor of cysteinyl leukotriene-receptor activity.

14. A method of treating a subject having a disorder that is responsive to cysteinyl leukotriene-receptor modulation, which method comprises administering to said subject a therapeutically effective amount of a substance according to claim 12 or 13.

15. A method according to claim 14 wherein the disorder is asthma, chronic obstructive pulmonary disease (COPD), allergic rhinitis, cardiac arrhythmia, myocardial ischaemia, atherosclerosis or heart failure.

16. Use of a substance as defined in claim 12 or 13 in the manufacture of a medicament for treatment or prophylaxis of a disorder that is responsive to stimulation or modulation of cysteinyl leukotriene-receptor activity.

17. A use according to claim 16 wherein the disorder is asthma, chronic obstructive pulmonary disease (COPD), allergic rhinitis cardiac arrhythmia, myocardial ischaemia, atherosclerosis and heart failure

18. A method of producing a polypeptide according to any one of claims 1 to 3 which method comprises maintaining a host cell as defined in claim 7 under conditions suitable for obtaining expression of the polypeptide and isolating the said polypeptide.


SEQUENCE LISTING (2 sequences)

SEQ ID NO: 1 - DNA, 1039 bp, Homo sapiens
  CDS (1)..(1038)
atggagagaaaatttatgtccttgcaaccatccatctccgtatcagaaatggaaccaaatggcaccttcagcaataacaacagcaggaactgcacaattgaaaacttcaagagagaatttttcccaattgtatatctgataatatttttctggggagtcttgggaaatgggttgtccatatatgttttcctgcagccttataagaagtccacatctgtgaacgttttcatgctaaatctggccatttcagatctcctgttcataagcacgcttcccttcagggctgactattatcttagaggctccaattggatatttggagacctggcctgcaggattatgtcttattccttgtatgtcaacatgtacagcagtatttatttcctgaccgtgctgagtgttgtgcgtttcctggcaatggttcacccctttcggcttctgcatgtcaccagcatcaggagtgcctggatcctctgtgggatcatatggatccttatcatggcttcctcaataatgctcctggacagtggctctgagcagaacggcagtgtcacatcatgcttagagctgaatctctataaaattgctaagctgcagaccatgaactatattgccttggtggtgggctgcctgctgccatttttcacactcagcatctgttatctgctgatcattcgggttctgttaaaagtggaggtcccagaatcggggctgcgggtttctcacaggaaggcactgaccaccatcatcatcaccttgatcatcttcttcttgtgtttcctgccctatcacacactgaggaccgtccacttgacgacatggaaagtgggtttatgcaaagacagactgcataaagctttggttatcacactggccttggcagcagccaatgcctgcttcaatcctctgctctattactttgctggggagaattttaaggacagactaaagtctgcactcagaaaaggccatccacagaaggcaaagacaaagtgtgttttccctgttagtgtgtggttgagaaaggaaacaagagtat

SEQ ID NO: 2 - protein, 346 aa, Homo sapiens
MERKFMSLQPSISVSEMEPNGTFSNNNSRNCTIENFKREFFPIVYLIIFFWGVLGNGLSIYVFLQPYKKSTSVNVFMLNLAISDLLFISTLPFRADYYLRGSNWIFGDLACRIMSYSLYVNMYSSIYFLTVLSVVRFLAMVHPFRLLHVTSIRSAWILCGIIWILIMASSIMLLDSGSEQNGSVTSCLELNLYKIAKLQTMNYIALVVGCLLPFFTLSICYLLIIRVLLKVEVPESGLRVSHRKALTTIIITLIIFFLCFLPYHTLRTVHLTTWKVGLCKDRLHKALVITLALAAANACFNPLLYYFAGENFKDRLKSALRKGHPQKAKTKCVFPVSVWLRKETRV